(19) United States
(12) Patent Application Publication
(10) Pub. No.: US 2022/0280066 A1
(43) Pub. Date: Sep. 8, 2022

(54) WEARABLE STRAIN SENSOR FOR MEASURING RESPIRATION RATE AND VOLUME

(71) Applicants: THE REGENTS OF THE UNIVERSITY OF CALIFORNIA, Oakland, CA (US); MAKANI SCIENCE INC., lrvine, CA (US)

(72) Inventors: Michael Chu, Irvine, CA (US); William E. Saltzstein, Irvine, CA (US); Michelle Khine, Irvine, CA (US)

(21) Appl. No.: 17/760,608

(22) PCT Filed: Sep. 16, 2020
(86) PCT No.: PCT/US20/51099

§ 371 Date: Mar. 15, 2022

Related U.S. Application Data
(60) Provisional application No. 62/901,071, filed on Sep. 16, 2019.

(51) Int. Cl. A61B 5/091; A61B 5/08; A61B 5/00

(52) U.S. Cl. CPC A61B 5/091; A61B 5/0816; A61B 5/6832; A61B 2562/164; A61B 2562/227; A61B 2562/0261

(57) ABSTRACT

A wearable strain sensor for measuring respiration volume and respiration rate is described herein. The wearable strain sensor includes a flexible yet not stretchable connector that connects soft electronics to hard electronics. The flexible and non-stretch able connector removes stress/strain from the soft/hard interface, thereby preventing damage to sensor components and maintaining electrical connection.

### CROSS-REFERENCES TO RELATED APPLICATIONS

This application claims benefit of U.S. Provisional Application No. 62/901,071 filed Sep. 16, 2019, the specification of which is incorporated herein in their entirety by reference.

### STATEMENT REGARDING FEDERALLY SPONSORED RESEARCH OR DEVELOPMENT

This invention was made with government support under Grant No. 4U54HL119893-04 awarded by NIH-NHLBI. The government has certain rights in the invention.

### BACKGROUND OF THE INVENTION

### FIELD OF THE INVENTION

The present invention relates to wearable systems, in particular, to a wearable strain sensor capable of simultaneously measuring both respiration rate and volume.

### BACKGROUND ART

Chronic respiratory disease (CRD) is a growing global health and economic burden. Two common CRDs, asthma and chronic obstructive pulmonary disease (COPD), affect over 435 million people worldwide; moreover, they each have an estimated medical cost of 50 billion dollars per year. Fortunately, most CRDs can be well controlled or even cured with proper monitoring and care. Patients with a CRD should be mindful of their respiratory status, and any sudden changes in condition should be addressed immediately to prevent further exacerbations.

There are several methods for assessing general respiratory health. The most common methods are pulmonary function tests (PFTs) that range from simple spirometry, which can be used to assess a patient's air flow, to full body plethysmography used to assess lung volumes. Other methods include arterial blood sampling and diffusion capacity. While these evaluations are effective in assessing a patient's respiratory health at a specific point in time in a laboratory setting, they cannot continuously monitor a patient's respiratory state under normal daily environments. Moreover, PFTs such as spirometry require the patient to breathe maximally into a mouthpiece, a maneuver that is challenging, which makes these types of tests difficult to ensure accurate readings and are not suitable for long term use.

Within a clinical setting, continuous monitors can be used to track a patient's respiration so that any measured changes in breathing patterns can be used as markers for intervention or as data for diagnoses. Apart from intervention and diagnosis purposes, data acquired from continuous respiration can provide valuable information on a patient's respiratory health and recovery. Continuous respiration monitoring can be achieved through different methods. Respiratory inductive plethysmography (RIP) uses two inductive belts placed around the abdomen and rib cage to measure the changes in circumference during respiration. The respiration volume can be calculated by knowing the change in circumference of both locations. However, because the bands are bulky and prone to slippage, this technology does not lend itself to monitoring patients throughout the day in their native environments. Similar to RIP, the motion of the chest wall and abdomen can also be measured visually using cameras or depth sensors. Optoelectronic plethysmography (OEP) uses several cameras to monitor reflective markers placed on the torso of the subject. The 3D coordinates of each marker can be determined, and a topographic map of the torso can be generated over time. The change in the topography can then be used to calculate respiration volume and rate. Transthoracic impedance measurements have also been used to calculate respiration rate and volume by measuring the change in impedance of the torso between several electrodes during respiration.

While these methods can all accurately track respiration rate and volume, they are either cumbersome to wear or require constant line of sight access to the patient's entire torso, which limits their use to research or clinical settings. Researchers have developed modified RIP systems that are more portable; however, the devices are still large and cumbersome as RIP requires access to the entire circumference of the chest and abdomen. Active monitoring of a patient's vitals requires the device to move seamlessly with the patient and to have an unobtrusive wearable form-factor. Wearable respiration monitors developed for these purposes are therefore small and discrete, making application and wear easy for the patient. However, existing sensors only measure respiration rate but not volume. Mechanical based sensors, such as strain and capacitive, have been developed to record torso movement to calculate respiration rate. Acoustic based sensors have also been developed to listen to air moving through the airway, and the actual breath itself can also be monitored using breath sensors placed under the nose. However, there is currently no unobtrusive system with a wearable form factor that can measure both respiration volume as well as respiration rate.

Currently, it is very difficult to adhere soft electronics onto hard electronics because of the stiffness mismatch between the two components. This causes a lot of stress between the interfaces, ultimately breaking the electronic components on the soft electronic side. Traditional electrical connection solutions, such as solder, do not work because the materials are not compatible with heat and mechanical properties of the connection damages the deposited metallic film layers on the flexible materials. This damage creates mechanical stress and degrades the connection over use, which causes increased resistance and intermittent connections. Other connection techniques such as mechanical methods that bring the wire to the film layer under pressure are unreliable and damage the very thin deposited layers, again resulting in very poor connection reliability, degradation, and permanent damage to the sensor. The issues above have hindered industry adoption of metal deposition flexible sensors due to the absence of viable solutions. Hence, there exists a need for a wearable sensor capable of measuring both respiration volume and respiration rate and in which soft electronics can be connected to hard electronics without causing damage or affecting connection.

BRIEF SUMMARY OF THE INVENTION

It is an objective of the present invention to provide devices and methods of making said devices that allow for connection of soft electronics to hard electronics and measurement of respiration volume and respiration rate, as specified in the independent claims. Embodiments of the invention are given in the dependent claims. Embodiments of the present invention can be freely combined with each other if they are not mutually exclusive.

It is an objective of the present invention to prevent any large strain or stress concentration from forming at the interface between the stiff and soft material. In some aspects, the present invention features an electrical connection for connecting a sensor to a circuit, comprising a flexible connector attached to a sensor connection pad of the sensor, and a conductive connector attached to the flexible connector. The conductive connector is electrically coupled to the sensor connection pad and a circuit connection pad of the circuit. The electrical connection may be utilized in a sensor connection assembly.

In some embodiments, the sensor connection assembly comprises one or more sensor connection pads, a flexible connector coupled to the one or more sensor connection pads such that at least a portion of the one or more sensor connection pads is exposed through the flexible connector, one or more conductive connectors coupled to the flexible connector, where each conductive connector is contacting an exposed portion of the one or more sensor connection pads, and one or more circuit connection pads electrically coupled to the one or more conductive connectors. A non-limiting embodiment of sensor connection assembly is a wearable strain sensor having a flexible, but not stretchable, double-sided adhesive surrounding the interface between the stiff and soft material so that the stress/strain is not concentrated on the conductive interface. The interface itself does not experience any large strain.

In other aspects, the present invention provides a method of connecting a flexible and deformable sensor to an electronic circuit. The method may comprise attaching a flexible connector to a sensor connection pad of the flexible and deformable sensor such that at least a portion of the sensor connection pads is exposed through the flexible connector, attaching a conductive connector to the flexible connector such that the conductive connector is contacting the exposed portion of the sensor connection pad, and attaching a circuit connection pad of the circuit to the conductive connector.

In some embodiments, the flexible connector is a double-sided adhesive tape. In other embodiments, the flexible connector comprises a flexible membrane and an adhesive for attaching the sensor connection pad and the conductive connector to the flexible membrane. The flexible connector may be insulating. The flexible connector may be non-stretchable and/or incompressible.

In other embodiments, the conductive connector comprises a conductive adhesive, paste, or liquid. For example, the conductive connector may be a metallic adhesive, paste, or liquid such as silver epoxy or a conductive adhesive tape.

As used herein, a circuit refers to a conductive path or track through which electric current flows. A circuit can be disposed on a base substrate that may be flexible, somewhat flexible, or rigid/non-flexible. An example of a circuit is a flexible printed circuit (FPC). A circuit board, such as a printed circuit board (PCB), refers to a physical board base having multiple circuits and on which discrete electronic components can be mounted. The board base of the circuit board may be flexible, somewhat flexible, or rigid. The circuit is more rigid than the sensor, hence the circuit is referred to as “hard electronics” and the sensor is “soft electronics”, Attaching hard and soft electronics together leads to a mechanical mismatch at the hard-soft interface which is not robust and with enough strain, will fracture at the connection point.

One of the unique and inventive technical features of the present invention is the flexible but not stretchable connector, such as double-sided adhesive, used to create a stable interface between the soft electronic (strain sensor on elastomer) and the hard electronics (PCB board). Without wishing to limit the invention to any theory or mechanism, the double-sided adhesive helps protect the connection point between the soft electronic and hard electronic from mechanical stress and strain because the double-sided adhesive does not stretch. Also, the other important aspect is that the strain sensor is encapsulated in silicone, and that the only metal exposed is at the pads. This is important because the encapsulation layer acts as a buffer between the metal on the soft electronics, and the double-sided adhesive. This prevents the adhesive from damaging the metal and it also prevents a large amount of stress/strain from building up on the metal film by transferring stress concentration from the metal film to the stable interface. None of the presently known prior references or work has the unique inventive technical feature of the present invention. Furthermore, the technical feature of the present invention is counterintuitive. The reason that it is counterintuitive is because prior references teach away from the technical feature of the present invention. Prior references teach the distribution of stress at an interface between a flexible and deformable sensor and a hard material through the use of a gradual stiffness gradient to disperse said stress. The present invention implements the transference of stress concentration from one place to another WITHOUT dispersing said stress throughout the system. Thus, the present invention teaches away from the prior references and is counterintuitive.

In another aspect, the wearable strain sensor of the present invention includes silver epoxy used to fill through holes is a heatless way to electrically connect the soft electronics onto the wires. Using the silver epoxy alone, without the flexible double-sided adhesive, would not work because any mechanical movement results in stress and strain around the epoxy between the soft electrical component and the epoxy, which can cause cracks to form in those areas around the soft electronic components. The role of the flexible double-sided adhesive is to prevent movement from happening at the epoxy interface and to move the hard-soft interface (where the stress/strain will concentrate) to a non-critical area of the soft electronic. When the sensor is stretched, the stress/strain forms around the adhesive and the soft elastomer in an area that does not have direct contact to any sensitive electrical components, thus preventing cracking in the electrical components in those areas. Furthermore, the inventive technical features of the present invention contributed to a surprising result in that the sensor device was robust and worked really well.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

BRIEF DESCRIPTION OF THE SEVERAL VIEWS OF THE DRAWING(S)

The features and advantages of the present invention will become apparent from a consideration of the following detailed description presented in connection with the accompanying drawings in which:

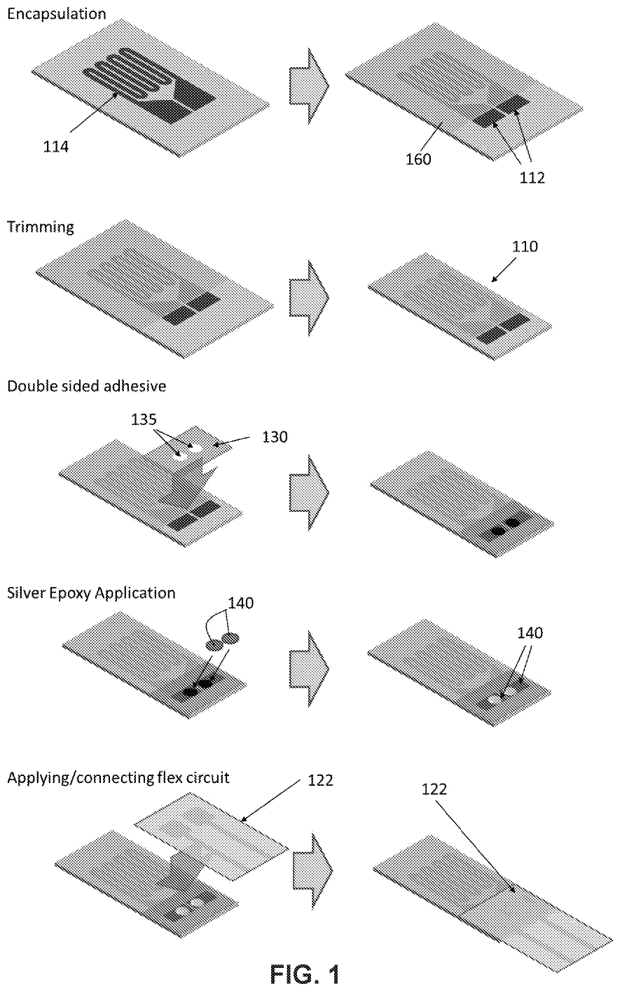

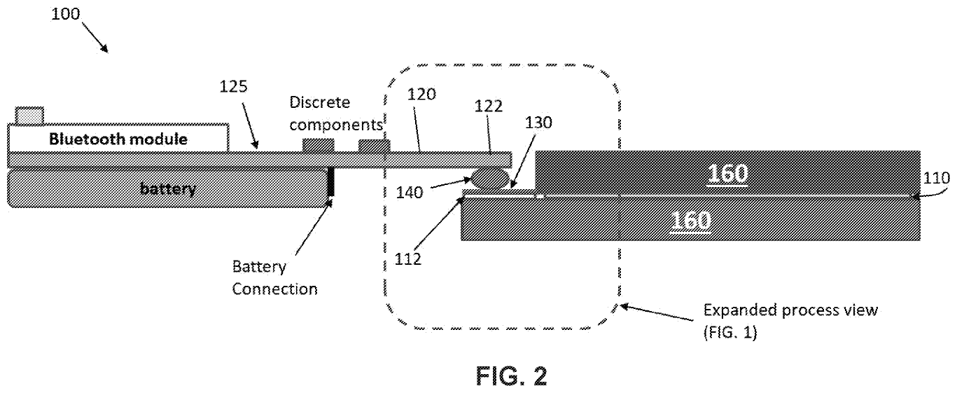

FIG. 1 is a flow diagram showing a method for connecting soft flexible and stretchable electronics to hard electronics.

FIG. 2 is a side view of a flex circuit connection used in a wearable strain sensor of the present invention.

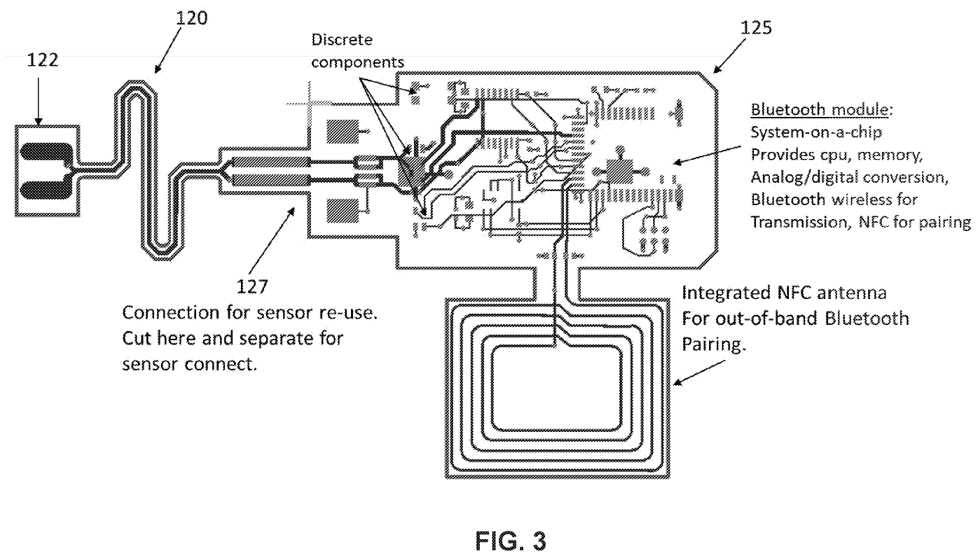

FIG. 3 shows a non-limiting embodiment of a circuit component of the wearable strain sensor.

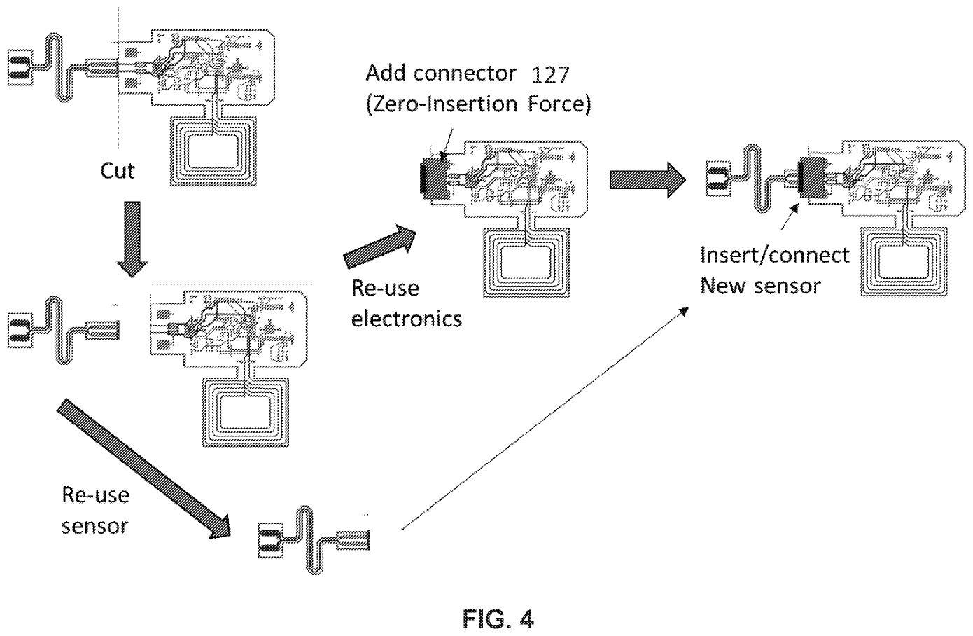

FIG. 4 is a flow diagram showing how a sensor connector and circuit can be separated and re-used to form a new sensor.

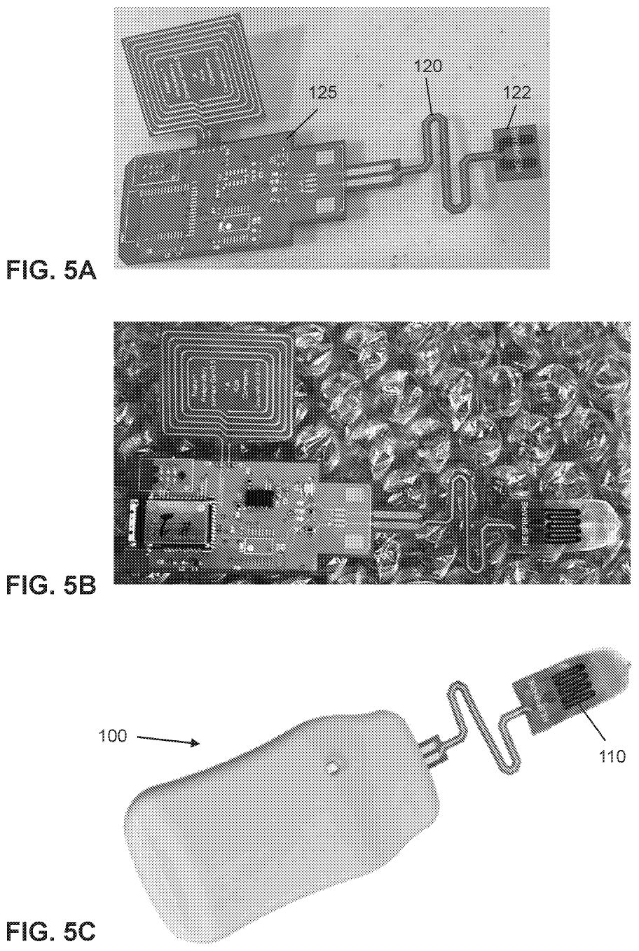

FIGS. 5A-5B show non-limiting embodiments of the circuit component.

FIG. 5C shows a non-limiting embodiment of the wearable strain sensor.

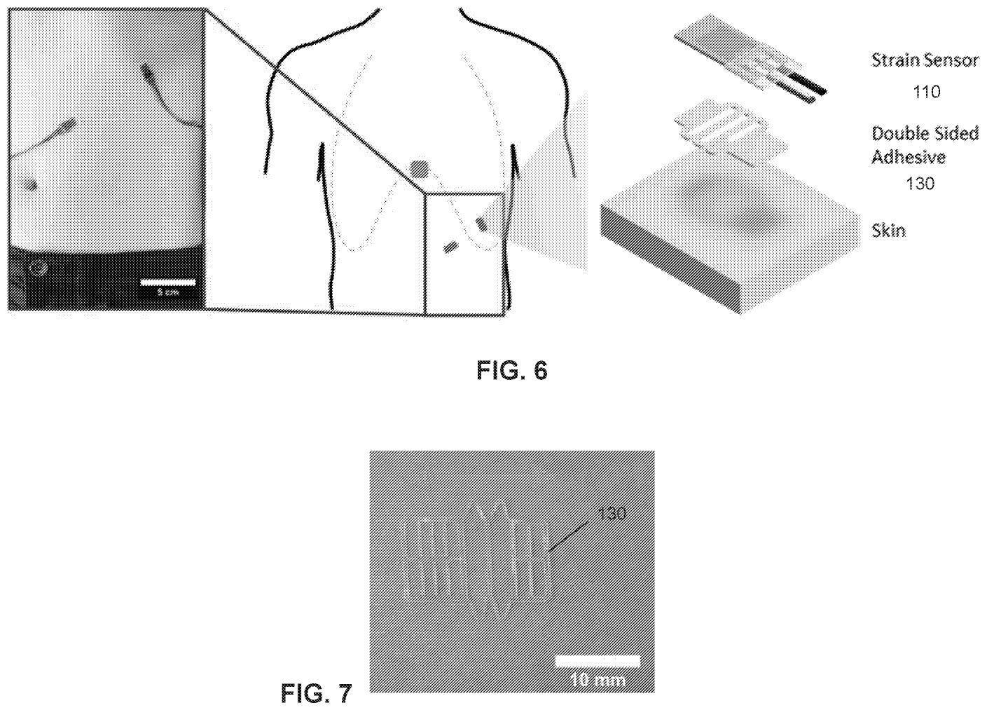

FIG. 6 shows the wearable strain sensor on the ribcage and abdomen. The middle schematic shows the placement of the accelerometer (purple square) in addition to the strain sensors (gray rectangles). The exploded schematic on the right shows the strain sensor and double-sided tape in order of attachment on the skin.

FIG. 7 shows a non-limiting embodiment of a double-sided tape with a curved laser cut strain relief structure.

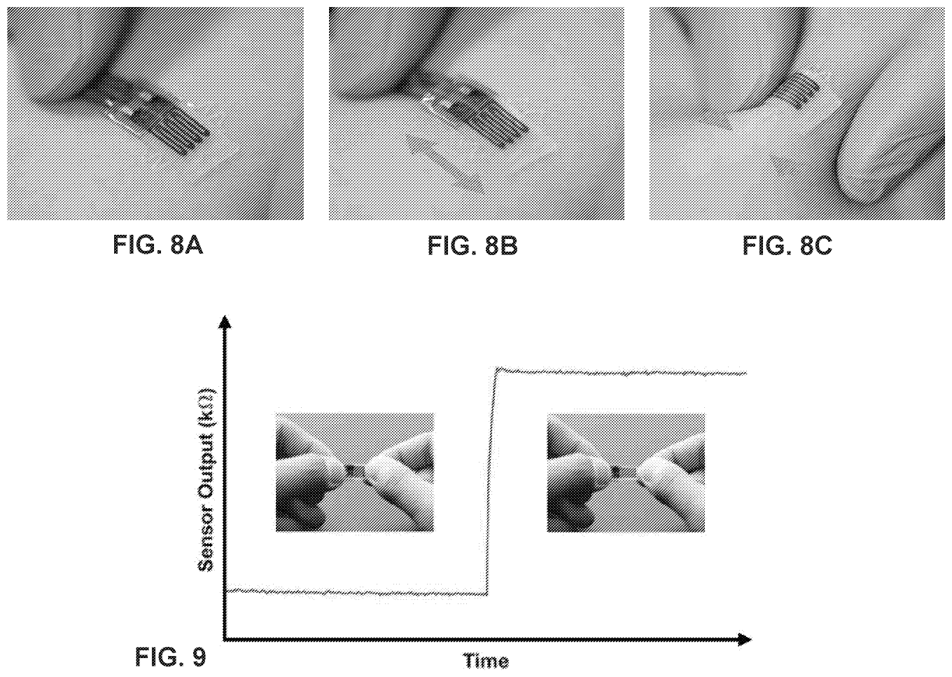

FIGS. 8A-8C show the strain sensor while neutral, under tension, and under compression, respectively. The arrow indicates direction of applied force.

FIG. 9 is a graph of a change in resistance of the sensor, under strain.

### DETAILED DESCRIPTION OF THE INVENTION

Following is a list of elements corresponding to a particular element referred to herein:

100 sensor device

110 sensor

112 sensor connection pad

114 sensing portion

120 circuit

122 circuit connection pad

125 circuit board

127 connector clip

130 flexible connector

135 apertures

140 conductive connector

160 elastomeric material

Referring now to FIGS. 1-9, the present invention features a sensor device (100) comprising a flexible and deformable sensor (110) having one or more sensor connection pads (112), a connecting region disposed on the one or more sensor connection pads (112), one or more conductive connectors (140) disposed on the connecting region, and a circuit board (125) having one or more circuit connection pads (122) connected to the one or more conductive connectors (140). The connecting region may comprise a flexible connector (130). The one or more conductive connectors (140) electrically couples the one or more circuit connection pads (122) to the one or more sensor connection pads (112), thereby forming an electrical connection between the flexible and deformable sensor (110) and the circuit board (125). In one embodiment, the flexible connector (130) includes one or more apertures (135) aligned with the one or more sensor connection pads (112) such that at least a portion of the one or more sensor connection pads is exposed through the one or more apertures (135). Preferably, each conductive connector (140) is contacting an exposed portion of the one or more sensor connection pads. In another embodiment, one circuit connection pad (122) is connected to one conductive connector (140). The flexible connector (130) may be non-stretchable and incompressible to maintain the electrical connection within an order of magnitude of parameters sensed by the sensor (110). The electrical connection may be maintained through at least 2500 cycles of physical movement. Stress may be concentrated at the connecting region instead of at the sensor (110) itself to allow for the electrical connection to be made between two dissimilar materials. For example, stress may be concentrated at a perimeter of the connecting region. In some embodiments, stress is not concentrated at a point of connection between the flexible connector (130) and the one or more conductive connectors (140). In some embodiments, the term “not concentrated” may refer to one or more degrees of magnitude lower in stress than a peak stress concentration.

In some embodiments, the flexible and deformable sensor (110) includes a sensing portion (114) juxtaposed between a top layer and a bottom layer of an elastomeric material (160) such that the sensing portion (114) is encapsulated by the elastomeric material. The elastomeric material (160) may be silicone. The elastomeric material (160) may aid in strengthening the flexible and deformable sensor (110) to allow for greater stress resistance. In one embodiment, the sensor may be a strain sensor comprising a piezo-resistive metal thin film set in a silicone elastomer substrate. The sensing mechanism is based on controlled fracturing of the metal thin film to increase resistance with respect to strain. The thin film itself has integrated hierarchical (nano- and micro-sized) wrinkle structures that not only act as strain relieving features but also help control the crack propagation, allowing the sensor to have a greater dynamic range while maintaining sensitivity.

In some embodiments, the flexible connector (130) is non-stretchable. In other embodiments, the flexible connector (130) is incompressible. In one embodiment, the flexible connector (130) is a double-sided adhesive substantially coplanar with the connection, comprising an insulating material with insulating properties in that plane. In some embodiments, the term “substantially coplanar” refers to 15 degrees or less of difference in angle between a first plane and a second plane. In another embodiment, the flexible connector (130) comprises a flexible membrane and an adhesive for attaching the one or more sensor connection pads (112) and the one or more conductive connectors (130) to the flexible membrane. The flexible connector (130) may comprise a non-conductive or insulating material. In some other embodiments, strain relief patterns may be cut into the flexible connector (130) to allow the sensor to stretch with the skin.

In other embodiments, the one or more conductive connectors (140) comprise a conductive adhesive, paste, or liquid. For example, the conductive connectors (140) may be a metallic adhesive, paste, or liquid. In one embodiment, the conductive connectors (140) comprise silver epoxy. In another embodiment, the conductive connectors (140) comprise a conductive adhesive.

In some embodiments, the sensor device (100) may further comprise a circuit (120) connecting the circuit board (125) to the one or more circuit connection pads (122). The circuit (120) is configured to mechanically isolate the flexible and deformable sensor (110) from the circuit board (125). The circuit (120) may be a flexible serpentine or looping circuit or a flexible wire. As shown in FIG. 4, the circuit (120) can be disconnected from the circuit board (125) and the flexible and deformable sensor (110). The circuit (120) can also be re-attached to the circuit board (125) and the flexible and deformable sensor (110). In one embodiment, the circuit (120) is re-attached to the circuit board (125) via a connector clip (127), such as a zero-insertion force connector.

Non-limiting embodiments of the sensor device are shown in FIGS. 5A-5C. The sensor device (100) may further include discrete components attached to the circuit board, such as a processing chip and power source (e.g. battery). The circuit board may also be encased in a housing. In further embodiments, the sensor device may further include a Bluetooth module for wireless respiration monitoring.

In some embodiments, the sensor device (100) of the present invention may be used to measure respiration rate and volume. For example, as shown in FIG. 6, the sensor device (100) may be attached to a subject's ribcage and abdomen to measure the expansion and contraction of the respective locations during respiration. Referring to FIGS. 8A-8C, double-sided, FDA approved, adhesive can be used to adhere the sensors to the skin. In one embodiment, the ends of the sensor can be anchored to the skin by the double-sided adhesive while a single strip of the adhesive is used to prevent the middle of the sensor from lifting off during respiration. Strain relieving structures may be used to allow the sensor and adhesive to strain in a single axis indicated by the blue arrow. The adhesive to the skin is an FDA approved skin adhesive while the adhesive to the sensor is a silicone-based adhesive.

According to other embodiments, the present invention provides methods of producing the sensor devices (100) described herein. In one embodiment, the method may comprise attaching a flexible connector (130) to one or more sensor connection pads (112) of a flexible and deformable sensor, attaching one or more conductive connectors (140) to the flexible connector (130), and attaching one or more circuit connection pads (122) of a circuit to the one or more conductive connectors (140). For example, one circuit connection pad (122) is connected to one conductive connector (140). In some embodiments, the flexible connector (130) includes one or more apertures (135) aligned with the one or more sensor connection pads (112) such that at least a portion of the one or more sensor connection pads is exposed through the one or more apertures (135). The conductive connectors (140) are attached to the flexible connector (130) such that each conductive connector (140) is contacting an exposed portion of the one or more connection pads.

In other embodiments, the method may further comprise encapsulating a sensing portion (114) of the flexible and deformable sensor in an elastomeric material (160), and curing the elastomeric material (160). The encapsulated flexible and deformable sensor (110) can be trimmed to remove excess elastomeric material (160) thereby achieving a desired shape and size.

In further embodiments, the method includes connecting the circuit (120) to a circuit board (125). A connector clip (127), such as a zero-insertion force connector, may be used to connect the circuit (120) to the circuit board (125). Preferably, the connector clip allows the circuit to be detached from and re-attached to the circuit board. The method disclosed herein is demonstrated in the following example.

### EXAMPLE

The following is a non-limiting example of the present invention. It is to be understood that said example is not intended to limit the present invention in any way. Equivalents or substitutes are within the scope of the present invention.

Strain sensor interface with flexible PCB (or other wire type material)

Referring to FIG. 1, a method for connecting soft flexible and stretchable electronics (based in silicone elastomer) to hard electrical wires is described herein. The stiff but flexible, double-sided adhesive used at the interface between the wire and the soft electronics helps eliminate a large concentration of stress and strain at the interface. This reduces the amount of cracking that could occur and increases the stability of the interface. The procedure for attaching the soft electronics onto the wires is as follows:

1. The sensing portion (114) of the strain sensor is encapsulated in silicone elastomer (160) leaving the two connection pads (112) exposed. The elastomer is allowed to cure.

2. The strain sensor (110) is trimmed to the proper size.

3. A double-sided adhesive (130) with exposed through-holes (135) in the same location as the connection pads (112) of the sensor is adhered onto the connection pad portion (112) of the strain sensor. The double-sided tape (130) must overlap onto the encapsulated portion of the sensor. The elastomer layer (160) helps protect the metal trace at the edge of the double-sided tape, preventing it from cracking. The adhesive adhered to the silicone side should ideally be some form of silicone based adhesive to produce the highest quality bond. There may be as many through holes (135) as needed depending on the number of connection pads for the given sensor.

4. The through-holes (135) of the double-sided tape will be filled with conductive silver epoxy (140). Other forms of conductive adhesive, paste, or liquid may be used.

5. Before the epoxy cures, the flexible PCB (125), or other type of electrical wire, is adhered onto the double-sided adhesive (130), such that the exposed metal wire is in contact with the silver epoxy (140) in the through hole (135) creating a conductive bridge to the pads (112) on the soft electronics. Any type of wires or conductive material can be used to attach onto the silver epoxy. The connections can be encapsulated with more tape or adhesive to strengthen the bond. In some embodiments, silver epoxy may be substituted or used in conjunction with any electrical based adhesive, paste, or liquid.

As used herein, the term “about’ refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. An electrical connection for connecting a flexible and deformable sensor (110) to a circuit (120), comprising a connecting region attached to a sensor connection pad (112) of the flexible and deformable sensor, and a conductive connector (140) attached to the connecting region, wherein the conductive connector is electrically coupled to the sensor connection pad (112) and a circuit connection pad (122) of the circuit, wherein the connecting region is non-stretchable and incompressible to maintain the electrical connection within an order of magnitude of parameters sensed by the sensor (110), wherein stress is concentrated at the connecting region.

2. The electrical connection of claim 1, wherein the connection region comprises a flexible connector (130).

3. The electrical connection of claim 2, wherein the flexible connector (130) is an adhesive material substantially coplanar with the connection, comprising an insulating material with insulating properties in that plane.

4. The electrical connection of claim 1, wherein the conductive connector (140) comprises a conductive adhesive, paste, or liquid.

5. The electrical connection of claim 4, wherein the conductive connector (140) comprises silver epoxy.

6. A sensor device (100) comprising:
a. a flexible and deformable sensor (110) having one or more sensor connection pads (112);
b. a flexible connector (130) disposed on the one or more sensor connection pads (112), wherein the flexible connector (130) includes one or more apertures (135) aligned with the one or more sensor connection pads (112) such that at least a portion of the one or more sensor connection pads is exposed through the one or more apertures (135);
c. one or more conductive connectors (140) disposed on the flexible connector (130), wherein each conductive connector (140) is contacting an exposed portion of the one or more sensor connection pads; and
d. a circuit board (125) having one or more circuit connection pads (122) connected to the one or more conductive connectors (140), wherein one circuit connection pad (122) is connected to one conductive connector (140),
wherein the one or more conductive connectors (140) electrically couples the one or more circuit connection pads (122) to the one or more sensor connection pads (112), thereby forming an electrical connection between the flexible and deformable sensor (110) and the circuit board (125),
wherein stress is not concentrated at a point of connection between the flexible connector (130) and the one or more conductive connectors (140).

7. The sensor device (100) of claim 6, wherein the sensor device (100) is configured to measure respiration rate and volume.

8. The sensor device (100) of claim 6, wherein the flexible connector (130) is a double-sided adhesive comprising an insulating material disposed substantially coplanar to the sensor connection pad (112).

9. The sensor device (100) of claim 6, wherein the flexible and deformable sensor (110) includes a sensing portion (114) juxtaposed between a top layer and a bottom layer of an elastomeric material (160) such that the sensing portion (114) is encapsulated by the elastomeric material.

10. The sensor device (100) of claim 9, wherein the elastomeric material (160) is silicone.

11. The sensor device (100) of claim 6, wherein the flexible connector (130) is non-stretchable and incompressible.

12. The sensor device (100) of claim 6, wherein the one or more conductive connectors (140) comprise a conductive adhesive, paste, or liquid.

13. The sensor device (100) of claim 6, wherein the one or more conductive connectors (140) comprise silver epoxy.

14. The sensor device (100) of claim 6, further comprising a circuit (120) connecting the circuit board (125) to the one or more circuit connection pads (122).

15. The sensor device (100) of claim 14, wherein the circuit (120) is a flexible conductor configured to mechanically isolate the flexible and deformable sensor (110) from the circuit board (125) configured to be disconnected from and re-attached to the circuit board (125) and the flexible and deformable sensor (110).

16. The sensor device (100) of claim 15, wherein the circuit (120) is re-attached to the circuit board (125) via a connector clip (127), wherein the connector clip (127) is a zero-insertion force connector.

17. A method of connecting a flexible and deformable sensor (110) to a circuit (120), comprising:
a. attaching a flexible connector (130) to a sensor connection pad (112) of the flexible and deformable sensor such that at least a portion of the sensor connection pads is exposed through the flexible connector (130);
b. attaching a conductive connector (140) to the flexible connector (130) such that the conductive connector (140) is contacting the exposed portion of the sensor connection pad; and
c. attaching a circuit connection pad (122) of the circuit to the conductive connector (140).

18. The method of claim 17, wherein the flexible connector (130) is non-stretchable and incompressible.

19. The method of claim 17, wherein the flexible connector (130) is a double-sided adhesive comprising an insulating material disposed substantially coplanar to the sensor connection pad (112).

20. The method of claim 17, wherein the conductive connector (140) comprises a conductive adhesive, paste, or liquid.

21. The method of claim 17, wherein the conductive connector (140) comprises silver epoxy.
